SEQUENCE LISTING (10 sequences)

SEQ ID NO: 1 - DNA, 69 bp, Artificial Sequence
  Synthetic construct
  misc_feature (25)..(27): n is a, c, g, or t
  misc_feature (30)..(33): n is a, c, g, or t
  misc_feature (36)..(39): n is a, c, g, or t
  misc_feature (42)..(44): n is a, c, g, or t
caagcagaagacggcatacgagatnnnatnnnncannnncgnnntggtcatgtggagacgctgggacag

SEQ ID NO: 2 - DNA, 24 bp, Artificial Sequence
  Synthetic construct
caagcagaagacggcatacgagat

SEQ ID NO: 3 - DNA, 25 bp, Artificial Sequence
  Synthetic construct
ctgtcccagcgtctccacatgacca

SEQ ID NO: 4 - DNA, 33 bp, Artificial Sequence
  Synthetic construct
gctgggacaggtcacttttcgtgcgccgcttca

SEQ ID NO: 5 - DNA, 25 bp, Artificial Sequence
  Synthetic construct
acactctttccctacattaactgca

SEQ ID NO: 6 - DNA, 18 bp, Artificial Sequence
  Synthetic construct
tctccgagcccacgagac

SEQ ID NO: 7 - DNA, 28 bp, Artificial Sequence
  Synthetic construct
tgggctcggagatgtgtataagagacag

SEQ ID NO: 8 - DNA, 24 bp, Artificial Sequence
  Synthetic construct
caagcagaagacggcatacgagat

SEQ ID NO: 9 - DNA, 20 bp, Artificial Sequence
  Synthetic construct
tcgtcggcagcgtcagatgt

SEQ ID NO: 10 - DNA, 56 bp, Artificial Sequence
  Synthetic construct
  misc_feature (19)..(21): n is a, c, g, or t
  misc_feature (24)..(27): n is a, c, g, or t
  misc_feature (30)..(33): n is a, c, g, or t
  misc_feature (36)..(38): n is a, c, g, or t
gaagacggcatacgagatnnnatnnnncannnncgnnngtctcgtgggctcggaga

(19) United States
(12) Patent Application Publication
(10) Pub. No.: US 2022/0325275 A1
(43) Pub. Date: Oct. 13, 2022

(54) Methods of Barcoding Nucleic Acid for Detection and Sequencing

(71) Applicant: Universal Sequencing Technology Corporation, Canton, MA (US)

(72) Inventors: Zhoutao Chen, Carlsbad, CA (US); Devin Porter, Carlsbad, CA (US); Guoya Mo, Vista, CA (US); Tsai-Chin Wu, San Marcos, CA (US)

(21) Appl. No.: 17/596,182

(22) PCT Filed: Jun. 4, 2020
(86) PCT No.: PCT/US2020/036198

§ 371 Date: Dec. 3, 2021

Related U.S. Application Data
(60) Provisional application No. 62/857,096, filed on Jun. 4, 2019.
(60) Provisional application No. 62/876,455, filed on Jul. 19, 2019.

(51) Int. Cl. C12N 15/10; C12Q 1/6869

(52) U.S. Cl. CPC C12N 15/1065; C12Q 1/6869

(57) ABSTRACT

The present invention provides methods to barcode nucleic acid for detection and sequencing. It applies a barcode template in a compartment with various targets, including nucleic acid fragments, nuclei and/or cells. After clonal amplification within the compartment, barcode sequence will integrate into its targets before the compartment is broken so that it will effectively barcode nucleic acid fragments originated from a nucleic acid fragment, a nucleus or a cell clonally. The barcode information can be used for tracking the origin of the fragment, nucleus or cell and be used for haplotype phasing and a variety of single cell-based applications N including whole genome sequencing, targeted sequencing, RNA sequencing and immune repertoire sequencing.

### RELATED APPLICATIONS

This application is a U.S. National Phase patent application under 35 U.S.C. 371 of International Application No. PCT/US2020/036198 filed Jun. 4, 2020, which claims priority to and the benefit of U.S. Provisional Application No. 62/857,096, filed on Jun. 4, 2019 and U.S. Provisional Application No. 62/857,096, filed on Jul. 19, 2019, the entire disclosures of each of which are hereby incorporated herein by reference.

### REFERENCE TO SEQUENCE LISTING

This specification includes a sequence listing submitted herewith, which includes the file entitled 168460-011201_ST25.txt having the following size: 3,007 bytes which was created Jun. 28, 2022, the contents of which are incorporated by reference herein.

### FIELD

The present invention relates in general methods for improved nucleic acid detection and sequencing.

### BACKGROUND

The present invention is in the technical field of genomics. More particularly, the present invention is in the technical field of nucleic acid sequencing. Nucleic acid sequencing can provide information for a wide variety of biomedical applications, including diagnostics, prognostics, pharmacogenomics, and forensic biology. Sequencing may involve basic low throughput methods including Maxam-Gilbert sequencing (chemically modified nucleotide) and Sanger sequencing (chain-termination) methods, or high throughput next-generation methods including massively parallel pyrosequencing, sequencing by synthesis, sequencing by ligation, semiconductor sequencing, and others. For most sequencing methods, a sample, such as a nucleic acid target, needs to be processed prior to introduction into a sequencing instrument. For example, a sample may be fragmented, amplified or attached to an identifier. Unique identifiers are often used to identify the origin of a particular sample. Most sequencing methods generate relatively short sequencing reads, ranging from tens of bases to hundreds of bases in length, and cannot generate complete haplotype phase information due to limited sequencing read length.

### SUMMARY

In one aspect, described herein are methods of tracking nucleic acid fragment origin by barcode tagging. The methods include providing a plurality of nucleic acid targets and a plurality of transpososomes, each transpososome comprises at least one transposon and one transposase; incubating nucleic acid targets and transpososomes together to form strand transfer complexes (STCs) on the nucleic acid targets; providing a plurality of unique barcode templates, wherein each barcode template comprises a central barcode sequence flanked by two handle sequences which can be used as priming site, hybridization site or binding site; compartmentalizing the nucleic acid targets with STCs and the barcode templates to generate two or more compartments comprise both nucleic acid targets and one barcode template or more than one barcode templates with different barcode sequences; amplifying the barcode template in the compartment, fragmenting nucleic acid target by breaking the STCs to form tagmented nucleic acid fragments, and attaching barcode sequence to tagmented nucleic acid fragments so that a plurality of fragments sharing the same one or more than one barcode sequence; removing the compartments and collecting the barcode tagged nucleic acid fragments.

In one aspect, described herein are methods of tracking nucleic acid fragment origin by barcode tagging. The methods include providing a plurality of nucleic acid targets and a plurality of transpososomes, each transpososome comprises at least one transposon and one transposase; incubating nucleic acid targets and transpososomes together to form strand transfer complexes (STCs) on the nucleic acid targets; providing a plurality of unique barcode templates, wherein each barcode template comprises a central barcode sequence flanked by two handle sequences which can be used as priming site, hybridization site or binding site; compartmentalizing the nucleic acid targets with STCs and the barcode templates to generate two or more compartments comprise both nucleic acid targets and one barcode template or more than one barcode templates with different barcode sequences; attaching a barcode sequence to the nucleic acid targets in the compartment by i) fragmenting nucleic acid target by breaking the STCs to form tagmented nucleic acid fragments; ii) amplifying the tagmented nucleic acid fragments with non-target-specific primers (i.e. only transposon specific), and amplifying the barcode template(s) at the same time; iii) linking a barcode template to a tagmented nucleic acid fragment, wherein a plurality of fragments sharing the same one or more than one barcode sequences; removing the compartments and collecting the barcode tagged nucleic acid fragments.

In one aspect, described herein are methods of single cell ATAC-seq. The methods include providing a plurality of nuclei or cells and a plurality of transpososomes, each transpososome comprises at least one transposon and one transposase; incubating them together to form strand transfer complexes (STCs) on accessible chromatin in the nuclei; providing a plurality of unique barcode templates, wherein each barcode template comprises a central barcode sequence flanked by two handle sequences which can be used as priming site, hybridization site or binding site; compartmentalizing the treated nuclei or cells and barcode templates to generate two or more compartments comprise both a nucleus or a cell and one barcode template or more than one barcode templates with different barcode sequences; amplifying the barcode template in the compartment, fragmenting accessible chromatin by breaking the STCs to form tagmented nucleic acid fragments, and attaching barcode sequence to tagmented nucleic acid fragments so that a plurality of fragments sharing the same one or more than one barcode sequences; removing the compartments and collecting the barcode tagged nucleic acid fragments; sequencing the barcode and barcode tagged nucleic acid to characterize the accessible chromatin region on a single cell basis.

In one aspect, described herein are methods of single cell ATAC-seq. The methods include providing a plurality of nuclei or cells and a plurality of transpososomes, each transpososome comprises at least one transposon and one transposase; incubating them together to form strand transfer complexes (STCs) on accessible chromatin in the nuclei; providing a plurality of unique barcode templates, wherein each barcode template comprises a central barcode sequence flanked by two handle sequences which can be used as priming site, hybridization site or binding site; compartmentalizing the treated nuclei or cell and barcode templates to generate two or more compartments comprise both a nucleus or a cell and one or more than one barcode templates with different barcode sequences; attaching a barcode sequence to accessible chromatin fragments in the compartment by i) fragmenting accessible chromatin by breaking the STCs to form tagmented nucleic acid fragments; ii) amplifying the said tagmented nucleic acid fragments and amplifying the barcode template at the same time; iii) linking a barcode template to a tagmented nucleic acid fragment, wherein a plurality of fragments sharing the same one or more than one barcode sequences; removing the compartments and collecting the barcode tagged nucleic acid fragments; sequencing the barcode and barcode tagged nucleic acid to characterize the accessible chromatin region on a single cell basis.

In one aspect, described herein are methods of barcoding a genome of a single cell. The methods include providing a plurality of nuclei or cells and treating them to expose DNA from chromatin by denaturing the proteins on the chromatin while keeping the cellular unit intact; providing a plurality of transpososomes, each transpososome comprises at least one transposon and one transposase; incubating treated nuclei or cells and the transpososomes together to form strand transfer complexes (STCs) on double stranded nucleic acid in the nuclei or cells; providing a plurality of unique barcode templates, wherein each barcode template comprise a central barcode sequence flanked by two handle sequences which can be used as priming site, hybridization site or binding site; compartmentalizing the treated nuclei or cells and barcode templates to generate two or more compartments comprise both a nucleus or a cell and one or more than one barcode templates with different barcode sequences; amplifying the barcode template in the compartment, breaking the STCs to form tagmented nucleic acid fragments, and attaching barcode sequence to tagmented nucleic acid fragments so that a plurality of fragments sharing the same barcode sequence; removing the compartments and collecting the barcode tagged nucleic acid fragments.

In one aspect, described herein are methods of barcoding a genome of a single cell. The methods include providing a plurality of nuclei or cells and treating the nuclei or cells to expose DNA from chromatin by denaturing the proteins on the chromatin while keeping the cellular unit intact; providing a plurality of transpososomes, each transpososome comprises at least one transposon and one transposase; incubating treated nuclei or cells and the transpososomes together to form strand transfer complexes (STCs) on double stranded nucleic acid in the nuclei or cells; providing a plurality of unique barcode templates, wherein each barcode template comprise a central barcode sequence flanked by two handle sequences which can be used as priming site, hybridization site or binding site; compartmentalizing the treated nuclei and barcode templates to generate two or more compartments comprise both a nucleus or a cell and one or more than one barcode templates with different barcode sequences; attaching a barcode sequence to said genomic DNA in said nucleus or cell in the compartment by i) breaking the STCs to form tagmented nucleic acid fragments; ii) amplifying the said tagmented nucleic acid fragments and amplifying the barcode template at the same time; iii) linking a barcode template to a tagmented nucleic acid fragment, wherein a plurality of fragments sharing the same one or more than one barcode sequences; removing the compartments and collecting the barcode tagged nucleic acid fragments.

In one aspect, described herein are methods for single cell targeted sequencing. The methods include providing a plurality of cells or nuclei, providing a plurality of unique barcode templates, wherein each barcode template comprise a central barcode sequence flanked by two handle sequences which can be used as priming site, hybridization site or binding site, and providing a plurality of target specific primers, wherein said target specific primers is capable of attaching to barcode templates; compartmentalizing the cells or nuclei, the barcode templates and the target specific primers to generate two or more compartments comprise a said cell or nucleus, one or more than one barcode templates with different barcode sequences, and target specific primers; amplifying the barcode template in the compartment, attaching the barcode sequence to the target specific primers, priming target genomic regions with target specific primers to generate barcode attached target fragments so that a plurality of barcode attached target fragments sharing the same one or more than one barcode sequences; removing the compartments and collecting the barcode attached target fragments; and sequencing the barcode and barcoded tagged nucleic acid to characterize the targeted regions on a single cell basis.

In one aspect, described herein are methods for single cell targeted sequencing. The methods include providing a plurality of cells or nuclei, providing a plurality of unique barcode templates, wherein each barcode template comprise a central barcode sequence flanked by two handle sequences which can be used as priming site, hybridization site or binding site, and providing a plurality of target specific primers, wherein said target specific primers is capable of attaching to barcode templates; compartmentalizing the cells or nuclei, the barcode templates and the target specific primers to generate two or more compartments comprise a said cell or nucleus, one or more than one barcode templates with different barcode sequences and target specific primers; attaching a barcode sequence to a targeted nucleic acid fragment in the compartment by i) breaking cell or nuclear membrane to release nucleic acids; ii) amplifying the nucleic acid targets and amplifying the barcode template at the same time; iii) linking a barcode template to a said amplified nucleic acid target, wherein a plurality of nucleic acid targets sharing the same one or more than one barcode sequences; removing the compartments and collecting the barcode attached target fragments; and sequencing the barcode and barcoded tagged nucleic acid to characterize the targeted regions on a single cell basis.

In one aspect, described herein are methods for single cell RNA sequencing. The methods include providing a plurality of cells or nuclei, providing a plurality of unique barcode templates, wherein each barcode template comprise a central barcode sequence flanked by two handle sequences which can be used as priming site, hybridization site or binding site, providing a reverse transcriptase and providing a plurality of primers, wherein the primers are capable as primers for cDNA synthesis, or for barcode template amplification, or for priming with cDNA, or for a combination thereof; compartmentalizing the cells and/or nuclei, the barcode templates, the reverse transcriptase and the primers to generate two or more compartments comprise a cell and/or nucleus, one or more than one barcode templates with different barcode sequences, reverse transcriptase and primers; in the compartment, lysing the cell and/or nucleus, generating cDNAs, amplifying the barcode template, attaching said barcode sequence to cDNA fragments or fragments generated from cDNA so that a plurality of barcode attached fragments sharing the same one or more than one barcode sequences; removing the compartments and collecting the barcode attached fragments; and sequencing the barcode and barcoded tagged nucleic acid to characterize cDNA profile on a single cell basis. In one aspect, cells or nuclei are treated with a reverse transcriptase for cDNA synthesis before compartmentation.

### BRIEF DESCRIPTION OF THE DRAWINGS

FIG. 1 illustrates a nucleic acid barcoding method using transpososomes and barcode templates with compartmentation reaction. BC means barcode.

FIG. 2 illustrates methods to attach clonally amplified barcode template to tagmented nucleic acid fragment. BC means barcode. In a compartment, A) Barcode templates attach to tagmented fragment directly; B) Barcode templates attached to tagmented fragment indirectly via a linker oligonucleotide; C) Both barcode templates and tagmented fragments are amplified and barcode templates attached to tagmented fragments; D) Barcode templates with different barcode sequences and tagmented fragments are amplified and barcode templates attached to tagmented fragments.

FIG. 3 illustrates a single cell ATAC-seq library preparation method using transpososomes tagged nuclei and barcode templates with compartmentation reaction.

FIG. 4 illustrates a single cell whole genome barcoding method using transpososomes tagged alcohol-fixed nuclei and barcode templates with compartmentation reaction.

FIG. 5 illustrates a method to enrich targeted regions using barcoded nucleic acid fragments and target specific primer set.

FIG. 6 illustrates that barcoded single cell can significantly improve detection power of somatic mutation with the combined ability for individual cell identification and sequencing error correction with unique molecule identification (UMI).

FIG. 7 illustrates a single cell nucleic acid barcoding reaction for targeted sequencing in a compartment.

FIG. 8 illustrates clonal barcoding reactions in a droplet through dual amplification of barcode template(s) and tagmented fragments and attaching amplified barcode templates to tagmented fragments.

FIG. 9 illustrates sequencing read count histogram of same barcode Read 1 read distance to the next Read 1 alignment to demonstrate a linked-read feature.

FIG. 10 shows a TapeStation high sensitivity D1000 screen tape profile of a cleaned up single cell ATAC-seq library.

FIG. 11 shows a screen shot of Cell Ranger analysis report of a single cell ATAC-seq experiment.

Transposases in all the figures are illustrated as a tetramer in the transpososome based on the MuA transposition system.

### DETAILED DESCRIPTION

Most commercially available sequencing technologies have limited sequencing read length. Second generation high throughput sequencing technologies can sequence only several hundred bases and rarely reach a thousand bases. However, nucleic acid sequences of a gene can span from several kilobases to tens and hundreds of kilobases, which means sequencing read length of tens of kilobases is necessary to successfully determine the haplotypes of all genes.

Meanwhile, most sequencing today are bulk sequencing of DNA or RNA extracted from many cells at once although individual cells are different. By using averaged molecular or phenotypic measurements of a cell population to represent an individual cell behavior, conclusions could be biased by the expression profiles of a majority group of cells or over-expressed outliers; and we will not have the sensitivity to identify all unique patterns from an individual cell which could be distinctive functional behaviors for a cell at a given location and time. In addition, early tumor detection has been significantly restrained by limited ability to detect very low frequent somatic mutation currently due to presence of high background wild type signal from normal cells or tissue. However, with improved ability to identify every single cell, we will be able to separate the mutant tumor cells from wild type cells by genotyping at single cell level. This will remove the wild type background signal generated from normal cells almost completely and make somatic mutation detection as easy as germline mutation detection.

Both Tn5 transpososome and MuA transpososome have been previously described to simultaneously fragment DNA and introduce adaptors at high frequency in vitro, creating sequencing libraries for next-generation DNA sequencing (Adey et al 2010, Caruccio et al 2011, and Kavanagh et al 2013). These specific protocols remove any phasing or contiguity information because of the fragmentation of the DNA. In these protocols after DNA reaction with transpososomes, a column purification, a heat treatment step, a protease treatment or an incubation with SDS solution or EDTA solution was necessary to release the transposase from the strand transfer complexes (STC) so that DNA is tagmented into fragments. It has been known that MuA transpososome can form a very stable STC when attack DNA targets (Surette et al 1987, Mizuuchi et al 1992, Savilahti et al 1995, Burton and Baker 2003, Au et al 2004). Similar stability has also been observed for Tn5 transpososome during transposition reaction (Amini et al 2014).

This invention takes advantage of the stability of STC and clonal barcode generation by compartmentation amplification and provides methods to uniquely barcode nucleic acid targets sub-fragments and/or barcode nucleic acid in a single cell.

The term “adaptor” as used herein refers to a nucleic acid sequence that can comprise a primer binding sequence, a barcode, a linker sequence, a sequence complementary to a linker sequence, a capture sequence, a sequence complementary to a capture sequence, a restriction site, an affinity moiety, unique molecular identifier, and a combination thereof.

A “barcode template”, which contains a barcode sequence, flanked by at least one handle sequence at one end or two handle sequences at both ends. Length of barcode sequence ranges from 4 bases to 100 bases. The handle sequences can be used as binding sites for hybridization or annealing, as priming sites during amplification, or as binding site for sequencing primers or transposase enzyme. Furthermore, barcode sequences can be selected from a pool of known nucleotide sequences or randomly chosen from randomly synthesized nucleotide sequences.

The term “transposase” as used herein refers to a protein that is a component of a functional nucleic acid protein complex capable of transposition and which is mediating transposition, including but not limited to Tn, Mu, Ty, and Tc transposases. The term “transposase” also refers to integrases from retrotransposons or of retroviral origin. It also refers to wild type protein, mutant protein and fusion protein with tag, such as, GST tag, His-tag, etc. and a combination thereof.

The term “transposon”, as used herein, refers to a nucleic acid segment that is recognized by a transposase or an integrase and is an essential component of a functional nucleic acid-protein complex capable of transposition. Together with transposase they form a transpososome and perform a transposition reaction. It refers to both wild type and mutant transposon.

A “transposable DNA” as used herein refers to a nucleic acid segment that contains at least one transposon unit. It can also comprise an affinity moiety, un-natural nucleotides, and other modifications. The sequences besides the transposon sequence in the transposable DNA can contain adaptor sequences.

The term “transpososome” as used herein refers to a stable nucleic acid and protein complex formed by a transposase non-covalently bound to a transposon. It can comprise multimeric units of the same or different monomeric unit.

A “transposon joining strand” as used herein means the strand of a double stranded transposon DNA that is joined by the transposase to the target nucleic acid at the insertion site.

A “transposon complementary strand” as used herein means the complementary strand of the transposon joining strand in the double stranded transposon DNA.

A “strand transfer complex (STC)” as used herein refers to a nucleic acid-protein complex of transpososome and its target nucleic acid into which transposons insert, wherein the 3′ ends of transposon joining strand are covalently connected to its target nucleic acid. It is a very stable form of nucleic acid and protein complex and resists extreme heat and high salt in vitro (Burton and Baker, 2003).

A “strand transfer reaction” as used herein refers to a reaction between a nucleic acid and a transpososome, in which stable strand transfer complexes form.

A “reaction vessel” as used herein means a substance with a contiguous open space to hold liquid; it is selected from the group consisting a tube, a well, a plate, a well in a multi-well plate, a slide, a spot on a slide, a droplet, a tubing, a channel, a bottle, a chamber and a flow-cell.

A “tagmented fragment” as used herein means a nucleic acid fragment tagged with at least one transposon end after a strand transfer reaction with a transpososome.

Encapsulating Nucleic Acid with Strand Transfer Complexes and Barcode Templates in Water-In-Oil Emulsion Droplets

This invention provides a method to encapsulate nucleic acid targets with STCs and a barcode template in water-in-oil emulsion droplets, and further generate barcode tagged nucleic acid fragments.

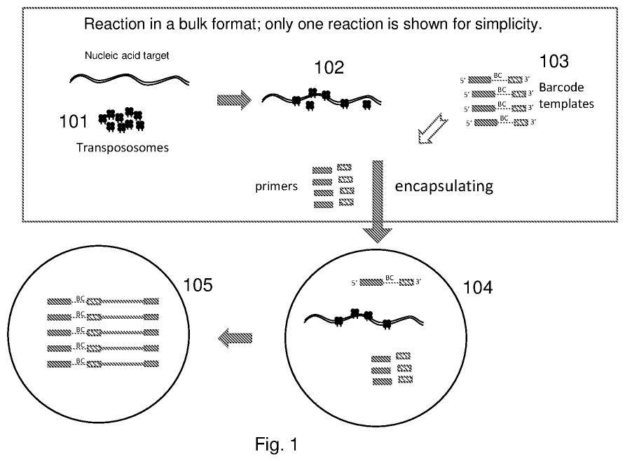

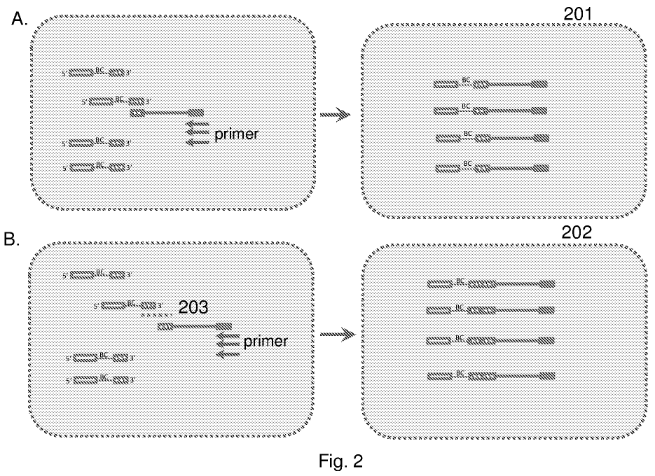

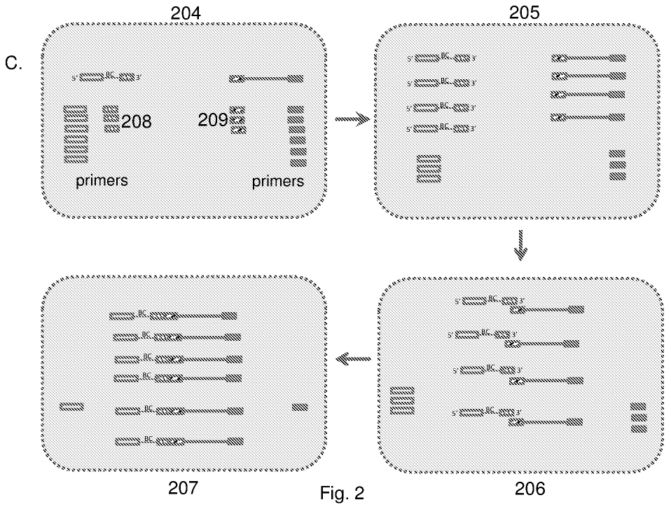

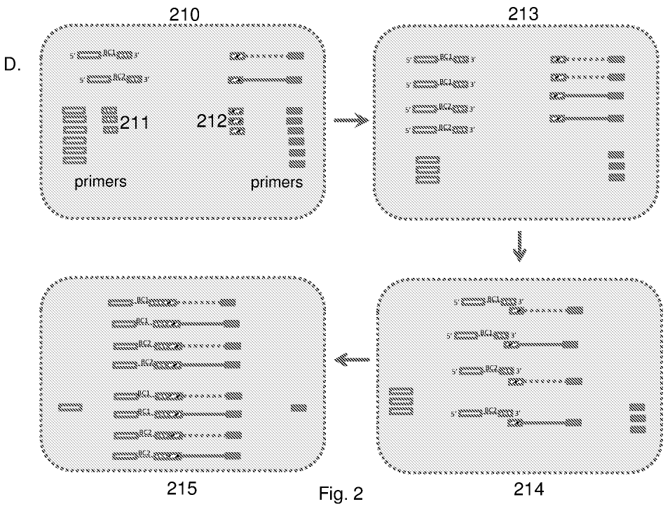

Nucleic acid targets are reacted with transpososomes (101) and form stable strand transfer complexes (102) while keep the contiguity of nucleic acid targets (FIG. 1). The nucleic acid targets are double-stranded. In some embodiment, they are double stranded DNA. In some embodiments, they are DNA and RNA hybrid. The strand transfer reactions happen with a plurality of nucleic acid targets in one reaction vessel. In some embodiment, one type of transpososome is used; in other embodiments, more than one types of transpososome are used simultaneously or sequentially. The nucleic acid targets with STCs (102) are mixed with a plurality of barcode templates (103) in the solution. In some embodiment, each barcode template has a unique barcode sequence and different from others. In some embodiment, a majority of barcode templates each has a unique barcode sequence and different from others. Anything greater than 50% can be considered as a majority. Preferably, greater than 90% barcode templates each has a unique barcode sequence and different from others. At least one of the transposable DNA in the transpososome is capable of hybridizing to one end of barcode template directly (FIG. 2A) or indirectly with a linker and/or a primer (FIG. 2B). Additional enzymes and substrates, such as, DNA polymerase, dNTP and primers are also provided in an aqueous solution in the same reaction vessel. In some embodiment, primers are used to amplify the barcode template. In some embodiment, primers can be used to amplify tagmented nucleic acid target fragments. Amplification includes exponential amplification and linear amplification. In some embodiment, different primers can be used to amplify the barcode template and tagmented nucleic acid target fragments in parallel (FIG. 2C), then the two groups of amplified products are capable to merge into one piece via shared homology between the two inner primers (FIG. 2C, 208 and 209) or via an additional linker which is capable to bridge a barcode template and a tagmented fragment together. Water-in-oil emulsion droplets (104) are generated. In some embodiment, one to a few nucleic acid targets with STCs are mixed with one barcode template in one droplet. Proper titration of emulsion droplets and barcode templates can be used here based on the Poisson distribution. In some embodiment, more than one barcode templates with different barcode sequences can be used in an emulsion droplet. It will significantly increase the barcode presence in the emulsion droplets and number of droplets with positive products so that it will increase the reaction yield significantly. In some embodiment, when both barcode templates and tagmented fragments are amplified before attaching a barcode sequence to a tagmented fragment, more than one barcode templates with different barcode sequences in the same emulsion droplet may not affect the true representation of the nucleic acid targets at all if different barcodes are randomly attached to the amplified copies of tagmented fragments (FIG. 2D). In this way, almost every emulsion droplet will contain at least a barcode template, which will be available for barcode attachment to a nucleic acid target when the target is also present in the droplet. This makes it feasible to get almost 100% droplets which contain any nucleic acid target be useful for reaction. The emulsion droplets have a diameter from 5 μm to 200 μm, and preferably from 5 μm to 50 μm. After a heat treatment, such as, at 60° C. to 75° C. for about 5-10 minutes, transposase will be released from the STCs and nucleic acid target breaks into smaller tagmented fragments. When still in a water-in-oil droplet, a DNA polymerase will fill in the gaps left during the transposition reaction. Emulsion amplification is performed to amplify barcode template in the droplet. Amplified barcode templates will hybridize to the tagmented fragments directly (FIG. 2A) or indirectly (FIG. 2B) and attach the barcode sequence to the fragments (105, 201, and 202) during amplification reaction. In some embodiment, unique molecular identifiers (UMIs) are added to the barcode templates during emulsion reaction. In some embodiment, UMIs are integrated as a linker (203) or a primer (209 and 212) in FIG. 2. In some embodiment, UMIs are introduced as a part of transposable DNA in the transpososomes to tagmented fragments. After emulsion amplification reaction, emulsion droplets are broken by detergent, alcohol, organic chemicals, high salt, or combination of these. Aqueous phase solution is collected. In some embodiment, one or more biotinylated primers are used so that amplified barcoded fragments can be pulled out easily with streptavidin beads. In some embodiment, one or more biotinylated dNTPs are used in the emulsion amplification. In some embodiment, primers with sample-specific barcode are used in the emulsion droplets during emulsion amplification so that emulsion amplification products from different sample reactions can be pooled together for final amplification or adaptor modification to make sequencing ready libraries.

In some embodiment, the nuclei acid targets are whole genomic DNA. This barcoding method can be used to generate long-range sequencing information for de novo sequencing, whole genome haplotype phasing and structural variant detection. In some embodiments, the nucleic acid targets are DNA fragments, cDNA, DNA/RNA hybrid, or a portion of captured DNA by hybridization capture, primer extension or PCR amplification. This barcoding method will be able to phase the variants in these molecules.

Encapsulating Transposase Tagged Nuclei and Barcode Template in Water-In-Oil Emulsion Droplets

This invention provides a method to encapsulate nuclei after strand transfer reaction and a barcode template in water-in-oil emulsion droplets, and further generate barcode tagged nucleic acid fragments for single cell level analysis.

ATAC-seq (Assay for Transposase-Accessible Chromatin using sequencing) is gaining more and more popularity as a state-of-the-art molecular biology tool to assess genome-wide chromatin accessibility (Buenrostro et al, 2013). ATAC-seq identifies accessible chromatin regions by tagging open chromatin with a hyperactive mutant Tn5 transposase that integrates sequencing adaptors into open regions of the genome. The tagged DNA fragments are purified, amplified by PCR and sequenced. Sequencing reads are then used to infer regions of increased accessibility as well as to map regions of transcription-factor binding sites and nucleosome positions. While natural wild type transposases have a low level of activity, ATAC-seq employs a mutated hyperactive transposase (Reznikoff et al, 2008), which has been successfully adapted to efficiently identify open chromatin and identify regulatory elements across the genome. Furthermore, single cell ATAC-seq is to separate single nuclei and perform ATAC-seq reactions individually (Buenrostro et al, 2015). Higher throughput single cell ATAC-seq uses combinatorial cellular indexing to measure chromatin accessibility in thousands of individual cells. Single-cell ATAC seq enables the identification of cell types and states for developmental lineage tracing. ATAC-seq will likely be a key component of comprehensive epigenomic workflows.

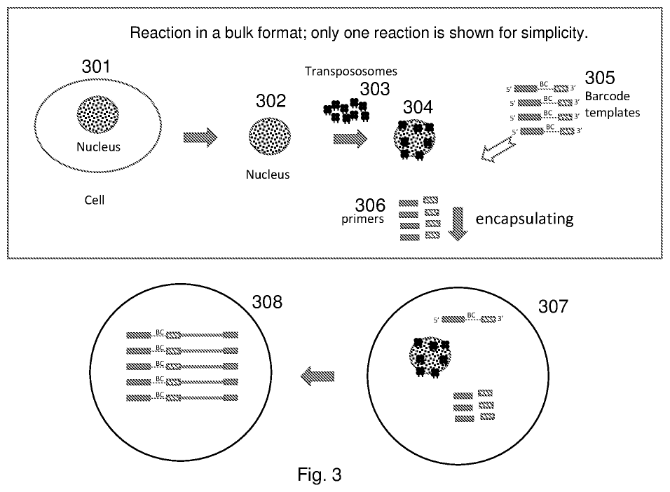

This invention uses emulsion method to encapsulate a transposase treated nucleus and a unique barcode template, then clonally amplify the barcode template within an emulsion droplet and attach the clonally amplified barcodes to tagmented accessible DNA fragments (FIG. 3). This barcoding method offers a high throughput and low-cost cellular indexing for single cell ATAC-seq analysis.

In some embodiment, nuclei (302) are collected from cells or tissue samples and incubated with transpososomes to form STCs (304), then mixed with a plurality of different barcode templates in a bulk reaction (FIG. 3). In some embodiment, whole cells are treated with transpososomes directly without nuclei isolation. In some embodiment, the transpososome comprises a mutated hyperactive TN5 transposase. In some embodiment, the transpsosome comprises a MuA transposase. Other enzymes and substrates, such as, DNA polymerase, dNTP and primers are also provided in an aqueous solution in the same bulk reaction. Water-in-oil emulsion droplets are generated. In some embodiments, one nucleus and one barcode template are present in most droplets by limiting titration or partitions based on Poisson distribution (307). 10× Genomics single cell ATAC-seq method used barcoded beads (GEMs) which comprise numerous copies of oligonucleotides functioning as barcode templates with the same barcode sequence in the emulsion droplet. This invention is to encapsulate only single copy of a barcode template without attaching to any physical carrier. In some embodiment, more than one barcode templates with different barcode sequences in an emulsion droplet are targeted to enable almost all the droplets contains at least one barcode template in order to increase the nucleus capture rate. The emulsion droplets have a diameter from 10 μm to 200 μm, and preferably from 20 μm to 100 μm. After a heat treatment, such as, at 60° C. to 75° C. for about 5-10 minutes, transposase will be released from the STCs and nucleic acid targets break into smaller tagged fragments. When still in a water-in-oil droplet, a DNA polymerase will fill in the gaps left during the transposition reaction on the tagged fragments. Nuclear membrane will break during emulsion PCR denaturing step, and emulsion amplification is performed to amplify barcode template in the droplet. Amplified barcode templates are capable to hybridize to the tagmented fragments directly or indirectly and attach the barcode sequence to the fragments during amplification reaction. In some embodiment, both barcoded templates and tagmented fragments are amplified parallelly first, then merged together to form barcoded tagmented fragments as FIGS. 2C and 2D. After emulsion amplification reaction, emulsion droplets are broken by detergent, alcohol, organic solution, high salt, or combination of these. Aqueous phase solution is collected. In some embodiment, one or more biotinylated primers or one or more biotinylated dNTPs are used so that amplified barcoded fragments can be pulled out easily with streptavidin beads. Sequencing library prepared from these barcoded fragments will be a single cell ATAC-seq library.

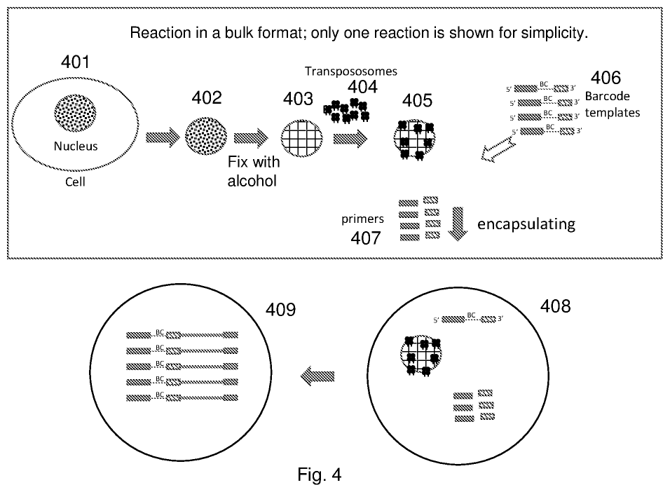

Besides single cell ATAC-seq application, this invention also provides a single cell whole genome sequencing method with proper modifications. It uses emulsion method to encapsulate a fixed nucleus treated with transposase and a unique barcode template, and clonally amplify the barcode template within an emulsion droplet and attach the barcodes to tagmented genomic DNA fragments (FIG. 4).

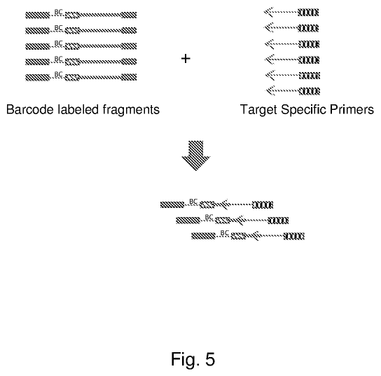

In some embodiment, nuclei (402) are collected from cells or tissue samples and fixed with alcohol-based fixation. Alcohol based fixative or other fixative will be able to denature the proteins in the nuclei but keep the nucleic acid intact. In this way, it will be able to expose all the genomic DNA from the chromatin. In some embodiment, fixed cells or tissue samples are used directly in the procedure without isolation of nuclei including the case for prokaryotic cells which lack a nucleus. After washing away fixation solution, nuclei are treated with transpososomes to form STCs (405) on the genomic DNA, then mixed with a plurality of different barcode templates in a bulk reaction. Other enzymes and substrates, such as, DNA polymerase, dNTP and primers are also provided in an aqueous solution in the same bulk reaction. Water-in-oil emulsion droplets are generated. In some embodiments, one nucleus and one barcode template are present in a droplet by limiting titration or partitions based on Poisson distribution (408). In some embodiment, more than one barcode templates with different barcode sequences in an emulsion droplet are targeted to enable almost all the droplets contains at least one barcode template in order to increase nucleus or cell capture rate. The emulsion droplets have a diameter from 10 μm to 200 μm, and preferably from 20 μm to 100 μm. After a heat treatment, such as, at 60° C. to 75° C. for about 5-10 minutes, transposase will be released from the STCs and nucleic acid target breaks into smaller tagmented fragments. When still in a water-in-oil droplet, a DNA polymerase will fill in the gaps left during the transposition reaction. Nuclear membrane will break during emulsion amplification. Emulsion amplification is performed to amplify barcode template in the droplet. Amplified barcode templates are capable to hybridize to the tagmented fragments directly or indirectly and attach the barcode sequence to the fragments during amplification reaction. In some embodiment, both barcoded templates and tagmented fragments are amplified parallelly first, then merged together to form barcoded tagmented fragments as FIGS. 2C and 2D. After emulsion amplification reaction, emulsion droplets are broken by detergent, alcohol, organic reagents, high salt, or combination of these. Aqueous phase solution is collected. In some embodiment, one or more biotinylated primers or one or more biotinylated dNTPs are used so that amplified barcoded fragments can be pulled out easily with streptavidin beads. In some embodiment, library prepared from these barcoded fragments can be used directly for single cell whole genome sequencing and single cell CNV analysis. In some embodiment, library prepared from these barcoded fragments can be used for further targeted capture of whole exome or smaller targeted region for targeted sequencing (FIG. 5).

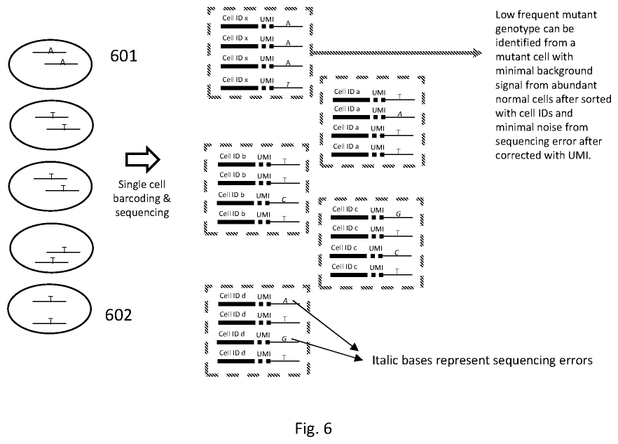

One advantage of this kind of single cell targeted sequencing is that it has much higher sensitivity for low frequent variant detection, such as, somatic mutation detection (FIG. 6). With the ability to uniquely barcoding individual cells, we can detect any mutations at a single cell level, which will effectively eliminate the background noise from surrounding cells. This enables very high sensitivity for detecting very low frequent somatic mutations which is required for early cancer detection. FIG. 6 illustrates the power of genotyping at a single cell level. There is a cell containing a mutant allele A (601), but there are many wild type cells containing a normal allele T (602) in the same sample. Unique molecular identifiers (UMIs) are added during emulsion reaction. In some embodiment, UMIs are integrated as a linker (203) or a primer (209 and 212) in FIG. 2. In some embodiment, UMIs are introduced as a part of transposable DNA in the transpososomes to tagmented fragments. With the incorporation of molecule specific UMI during single cell barcoding and sequencing, sequencing reads can be grouped based on their cell ID first, and for each cell, we are able to identify sequencing error based on UMI and make a correct variant call easily. This approach can be applied for circulating tumor cells, tissue biopsy samples or tissue sections. The tissue and/or sections include fresh frozen tissue/section and formalin-fixed paraffin-embedded tissue/section.

Encapsulating Cells, Barcode Templates and Target-Specific-Primers in Water-In-Oil Emulsion Droplets

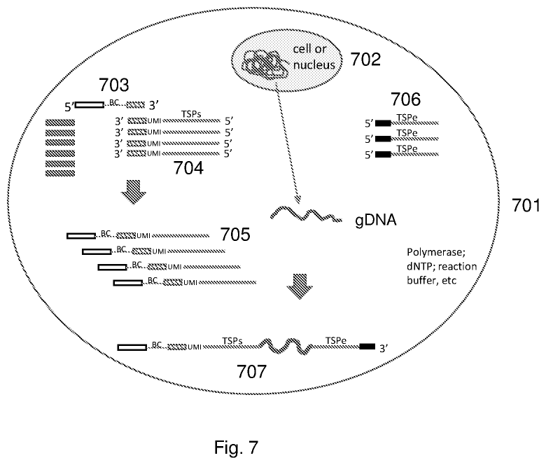

This invention provides a high throughput method for single cell targeted sequencing. Isolated cells or nuclei (702) are encapsulated with unique barcode templates (703) and first set of target specific primers (704) by emulsion droplets (FIG. 7). In some embodiments, cells or isolated nuclei are pretreated before encapsulation reaction. Pretreatment can be incubation with a fixative, in situ enzymatic reaction, hybridization, or a combination of these treatments. Additional enzymes and substrates, such as, DNA polymerase, dNTP and common primers are also provided in the aqueous solution. Water-in-oil emulsion droplets (701) are generated in such conditions that one cell or one nucleus and one barcode template are present in a droplet by limiting titration or partitions based on Poisson distribution. The emulsion droplets have a diameter from 10 μm to 200 μm, and preferably from 20 μm to 100 μm. Cell membrane or nuclear membrane will break during emulsion amplification and release genomic DNA into emulsion droplets. Emulsion amplification is performed to amplify barcode template and attach target specific primers to barcode template in the droplet. Single stranded amplified barcode templates with target specific sequences at 3′ end (705) are capable to hybridize to the genomic DNA targets and make copies of targeted region during amplification reaction. In some embodiment, a second set of target specific primers (706) are included in the aqueous solution during emulsion droplet generation. After emulsion amplification reaction, barcode tagged amplicons of the targets (707) will be generated, which can be used for sequencing library preparation and sequencing analysis. In some embodiment, to reduce primer dimers generated during amplification, dUTP containing primers can be used and in combination with UDG/APE1/ExoI treatment after emulsion amplification. Sequencing library adaptor can be added by ligation after cleaning up primer dimers.

In some embodiment, cell or nuclei are treated and reacted with a reverse transcriptase for in situ cDNA synthesis before encapsulating with emulsion droplets. In some embodiment, a reverse transcriptase and cDNA primers as the first set of primers can be included in the emulsion reaction. In some embodiment, cDNA primers have polyT sequence at the 3′ end; in some embodiment, cDNA primers have GGG at the 3′ end; in some embodiment, cDNA primers have target specific primers at the 3′ end. During the early phase of emulsion reaction, cDNA or partial cDNA will be generated from mRNA in the single cell or nucleus by reverse transcriptase. The barcoding reaction will proceed as described previously but use the cDNA as input DNA. With different primers used for reverse transcription or cDNA priming, this method can be modified for single cell transcriptome analysis, single cell RNA-Seq analysis, single cell target-seq application, and immune repertoire analysis.

In some embodiment, more than one barcode templates with different barcode sequences can be present in an emulsion droplet to increase the cell capture rate.

Cellular Indexing of Transcriptomes and Epitopes by Sequencing (CITE-seq) is a multimodal single cell phenotyping method, which uses DNA-barcoded antibodies to convert detection of proteins into a quantitative, sequencable readout. Antibody-bound oligos act as synthetic transcripts that are captured during most large-scale oligo dT-based single cell RNA-seq library preparation protocols (Stoeckius et al, 2017). For our method above, when cDNA primer is ployT type design, CITE-seq type library will be able to be generated efficiently.

There are many ways to generate water-in-oil emulsion, such as, by vortexing, homogenizing, filtering, pipetting, merging water and oil via microfluidic device, etc. In some embodiments, emulsification method for this invention is mixing aqueous solution and oil with a pipet in a microtube or well for ease-of-setup and scaleup of sample preparation procedures. Emulsion droplet size can be controlled by mixing speed and orifice size of the pipet tip. Proper sized emulsion droplets can be generated with a mixing velocity ranging from 20μl/5 to 1000μl/5.

Although the compartmentation method described in this invention is water-in-oil emulsion, other methods are also feasible. Certain type of liposomes, such as, giant unilamellar liposome vesicles (GUVs) with a size from 1-200 um in diameter, have showed very high thermostable and are able to perform PCR amplification inside of its enclosure (Kurihara et al 2011, Laouini et al 2012). In some embodiments, the emulsion droplets used for compartment generation in this invention can be replaced by GUVs. In some embodiments, the emulsion droplet used for compartment generation can be replaced by microwells, microarray, microtiter plate or other physically separated compartmentation methods.

Although the invention has been explained with respect to an embodiment, it is to be understood that many other possible modifications and variations can be made without departing from the spirit and scope of the invention as herein described.

Further, in general with regard to the processes, systems, methods, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments and should in no way be construed so as to limit the claimed invention.

Moreover, it is to be understood that the above description is intended to be illustrative and not restrictive. Many embodiments and applications other than the examples provided would be apparent to those of skill in the art upon reading the above description. The scope of the invention should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future embodiments. In sum, it should be understood that the invention is capable of modification and variation and is limited only by the following claims.

Lastly, all defined terms used in the application are intended to be given their broadest reasonable constructions consistent with the definitions provided herein. All undefined terms used in the claims are intended to be given their broadest reasonable constructions consistent with their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary.

### EXAMPLES

### Example 1. Barcoding Long Fragments in Droplets to Generate Linked Reads

This example describes a method of barcoding DNA fragments in droplets to generate linked reads.

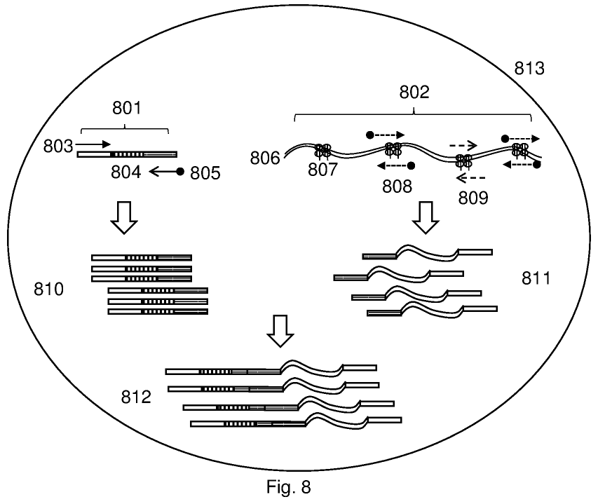

1 ng E. coli DH10b genomic DNA (FIG. 8, 806) was strand-transferred by incubating with a wild type transpososome and a mutant MuA transpososome (807) simultaneously using 14 of Barcoding Enyzme (wild type MuA transpososome) and 14 Tagging Enzyme (mutant MuA transpososome) from TELL-Seq WGS Library Reagent Box 1 (Universal Sequencing Technology, Carlsbad, Calif.) in 1× Reaction Buffer with Cofactor in 204 reaction volume at 37° C. for 15 minutes to form strand transfer complexes (STC, 802). Take 1 μL of STC reaction mixture into 104 of an amplification aqueous solution containing 1× Pfu polymerase buffer, dNTPs, barcode templates Code 1.2 (5′ CAAGCAGAAGACGGCATACGAGATNNNatNNNNcaNNNNcgNNNTGGTCATGTGGAG ACGCTGGGACAG-3′ (SEQ ID NO: 1), 801), primers [P7 (5′-CAAGCAGAAGACGGCATACGAGAT-3′ (SEQ ID NO: 2), 803), T25 (5′-CTGTCCCAGCGTCTCCACATGACCA-3′ (SEQ ID NO: 3), 804), tsMU (5′-GCTGGGACAGGTCACTTTTCGTGCGCCGCTTCA-3′ (SEQ ID NO: 4), 808), Bio-mP5 (5′-Biotin-ACACTCTTTCCCTACATTAACTGCA-3′ (SEQ ID NO: 5), 809)] and Pfu DNA polymerase in a 0.2 mL PCR tube. Add 904 of 7% Abil EM90 (Evonik Corporation, Richmond, Va.) in mineral oil (Sigma-Aldrich, St. Louis, Mo.). Set a P200 pipette at 704 and mix the solution by pipetting up and down for 30 times in 30 seconds. Transfer 504 mixture into another 0.2 mL PCR tube and add 504 of 7% Abil EM90 in mineral oil. Mix the solution by pipetting up and down for 15 times in 15 seconds. Perform amplification as following: 72° C. for 2 minutes, 94° C. for 30 seconds, 21 cycles of (94° C. for 20 seconds, 55° C. for 1 minute, 72° C. for 1 minute), 12 cycles of (94° C. for 30 seconds, 35° C. for 1 minute, 72° C. for 2 min), 72° C. for 3 minutes, hold at 4° C. At the end of PCR, add 100 μL of breaking buffer (100 mM NaCl, 10 mM Tris-HCl, pH 7.5, 0.2% SDS, 15% Isopropanol) and incubate for 10 minutes at room temperature. Spin the tube at 5,000 g for 10 minutes to separate oil and aqueous solution. Remove oil from the top layer. Transfer 704 of aqueous solution into a 0.5 mL low bind DNA tube and add 354 MyOne™ Streptavidin T1 beads (Life Technologies, Carlsbad, Calif.) in the binding buffer. Incubate at room temperature for 15 minutes with rotation. Wash the beads three times with bead wash buffer. Resuspend the beads in 154 of 0.02% Tween-20. Use 54 beads for PCR amplification in 404 total volume using Pfu DNA polymerase with P7 primer and one of multiplex primers from TELL-Seq Library Multiplex Primer (1-8) kit (Universal Sequencing Technology, Carlsbad, Calif.). Perform PCR amplification as following: 94° C. for 30 seconds, 6 cycles of (94° C. for 20 seconds, 58° C. for 1 minute, 72° C. for 1 minute), 72° C. for 3 minutes, hold at 4° C. After PCR amplification, clean up the library product with 0.9× AMPure XP beads and quantitate for sequencing. Different ratio of barcode template molecule to emulsion droplet was tested. 3 to 1 ratio was used in the example to make sure approximately 95% droplets containing at least one barcode template.

The library was sequenced in a 2×74 paired end run on a MiSeq system. The barcode templates used in the experiment contained 20-base barcode sequences and was sequenced as Index 1 read. Table 1 showed summary of the sequencing run. The mapping rates of read 1 and read 2 were 98.6% and 97.0%, respectively. Total 1,392,842 barcodes were identified.

TABLE 1
Sequencing Statistics on the E. coli library
from a 2 × 74 paired end MiSeq run

| Sequencing Metrics | Results |
|---|---|
| read_type | PE |
| read_length | 74 |
| reads_total | 7,921,891 |
| duplication rate | 17.81% |
| read1_reads_mapped_percentage | 98.6% |
| read2_reads_mapped_percentage | 97.0% |
| barcode_with_single_read | 316,297 |
| barcode_with_multi_reads | 1,281,011 |
| reads_related_to_barcode_with_multi_reads | 7,605,594 |
| barcode_corrected | 199,968 |
| error_barcode_number | 4,498 |
| final_correct_barcode_number | 1,392,842 |
| final_reads_number | 7,916,620 |

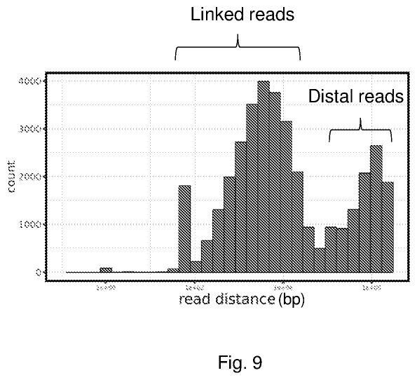

To examine if the barcoding reaction was clonal to the fragment tagged, we generated a read distance plot (FIG. 9) which was a Read 1 read count histogram of next alignment read distance for those R1 reads sharing the same barcode sequence. If the barcoding reaction was indeed clonal to the tagged fragment, there would be many same barcoded reads with short distance (less than 50 Kb usually) next to each other which would show as the linked reads population; while the same barcoded reads arising from different genomic DNA fragments would show much large distance (greater than 100 Kb usually) in the distal reads population. FIG. 9 showed very good clonal barcoding reaction for this E. coli library. We further de novo assembled these linked reads using TuringAssembler, which was a linked read assembler and got N50 contig size of 4,591,903 bp which was very close to the full size of an E. coli DH10B genome (4,686,137 bp) with very good assembly accuracy (Table 2).

TABLE 2
QUAST results of de novo assembly using TuringAssembler compared
with E. coli DH10B genome reference (4,686,137 bp)
de novo assembly results

| | |
|---|---|
| Genome fraction (%) | 99.054 |
| Duplication ratio | 1.068 |
| Largest alignment | 3,339,054 |
| Total aligned length | 4,950,726 |
| NG50 | 4,591,903 |
| NA50 | 3,339,054 |
| LG50 | 1 |
| LA50 | 1 |
| # mismatches per 100 kbp | 11.29 |
| # indels per 100 kbp | 1.53 |
| # N's per 100 kbp | 60.5 |

Statistics without reference

| | |
|---|---|
| # contigs | 159 |
| # contigs (>=0 bp) | 159 |
| # contigs (>=1000 bp) | 159 |
| # contigs (>=5000 bp) | 2 |
| # contigs (>=50000 bp) | 2 |
| Largest contig | 4,591,903 |
| Total length | 5,069,768 |
| Total length (>=0 bp) | 5,069,768 |
| Total length (>=1000 bp) | 5,069,768 |
| Total length (>=5000 bp) | 4,713,528 |
| Total length (>=50000 bp) | 4,713,528 |
| N50 | 4,591,903 |
| L50 | 1 |
| GC (%) | 50.71 |

### Example 2. Single Cell ATAC-Seq

K562 cells (ATCC, Manassas, Va.) were cultured in DMEM media (Life Technologies, Carlsbad, Calif.) with 10% FBS (Life Technologies, Carlsbad, Calif.), 1:100 MEM Non-Essential Amino Acids (Life Technologies, Carlsbad, Calif.), 1:100 Penicillin/Streptomycin (Life Technologies, Carlsbad, Calif.), 1:100 GlutaMax (Life Technologies, Carlsbad, Calif.), and 1:1000 BME (Life Technologies, Carlsbad, Calif.). When cells reached a concentration of about 500,000/mL, 1.5 million cells were added to a 1.5 mL protein low-bind centrifuge tube and centrifuged at 300×g for 3 minutes. The supernatant was removed, and the pellet was resuspended in 1 mL of 1×PBS. The cells were then centrifuged again at 300×g for 3 minutes. The cell pellet was resuspended in 1504 ice-cold lysis buffer (10 mM NaCl, 10 mM Tris pH 7.4, 3 mM MgCl₂, 0.01% digitonin, 0.1% tween, and 0.1% NP40). The cells were mixed 5× with a P200 pipette set to 100 μL and placed on ice for 3 minutes. After the 3-minute incubation, the cells were mixed 10 times with the pipette set at 100 μL. 8504 of wash buffer (10 mM NaCl, 10 mM Tris pH 7.4, 3 mM MgCl₂, 0.1% tween) was added and mixed 5 times with a P1000 pipette set at 8504. The nuclei were centrifuged at 400×g for 3 minutes and resuspended in 1 mL of wash buffer. The nuclei were filtered through a 0.4 μM flowmi filter to remove any clumps and then centrifuged again at 400×g for 3 minutes. The nuclei pellet was resuspended in 204 of wash buffer. 24 of nuclei was diluted in 984 and counted twice to obtain an accurate cell count. The final concentration was adjusted to 25,000 nuclei/4 and the nuclei were kept on ice.

5 μM Tn5ME transpososomes were assembled using EZ-Tn5™ Transposase (Lucigen, Middleton, Wis.) and preannealed Tn5MEDS-A and Tn5MEDS-B oligonucleotides (Picelli et al 2014). Strand transfer reaction was performed by treating 50,000 K562 nuclei with 0.35 μM Tn5ME transpososomes in a 204 reaction buffer (final 10% DMF, 10 mM Tris pH7.5, and 5 mM MgCl₂, 0.33×PBS, 0.1% tween, 0.01% digitonin). The mixture was incubated on a thermal cycler for 1 hour at 37° C. After the reaction, the nuclei were diluted to a final concentration of 500 nuclei/μL in nuclei resuspension buffer (10 mM NaCl, 10 mM Tris pH 7.4, 3 mM MgCl₂).

1,000 tagged nuclei were used in 204 of amplification mix comprising Pfu DNA polymerase, dNTP, primers [Tn5-BC-R (5′-TCTCCGAGCCCACGAGAC-3′)(SEQ ID NO: 6), Tn5-R2-F28 (5′-TGGGCTCGGAGATGTGTATAAGAGACAG-3′) (SEQ ID NO: 7), P7 (5′-CAAGCAGAAGACGGCATACGAGAT-3′) (SEQ ID NO: 8) and Tn5-R1-S (5′-TCGTCGGCAGCGTCAGATGT-3′) (SEQ ID NO: 9)], barcode template Code1.3 (5′-GAAGACGGCATACGAGATNNNatNNNNcaNNNNcgNNNGTCTCGTGGGCTCGGAGA-3′) (SEQ ID NO: 10) in a 0.2 mL PCR tube. 804 of an oil mixture [7% Abil EM90 (Evonik Corporation, Richmond, Va.) in mineral oil (Sigma-Aldrich, St. Louis, Mo.)] was added on top of the 204 amplification mixture. The targeted ratio of number of barcode templates to expected number of droplets was 3 to 1 in order to have approximately 95% of droplets containing at least one barcode template. Set a P200 pipette at 704 and mix the solution by pipetting up and down for 30 times in 45 seconds and additional 15 times in 30 seconds. The following PCR program was performed: 72° C. for 5 minutes, 95° C. for 30 seconds, 20 cycles of (95° C. for 15 seconds, 58° C. for 30 seconds, and 72° C. for 20 seconds), 5 cycles of (95° C. for 20 seconds, 40° C. for 2 minutes, and 72° C. for 30 seconds), 72° C. for 2 minutes, 20° C. for 1 minute, and hold at 4° C.

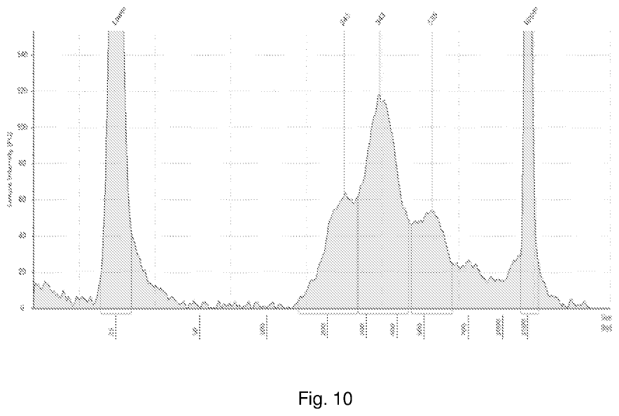

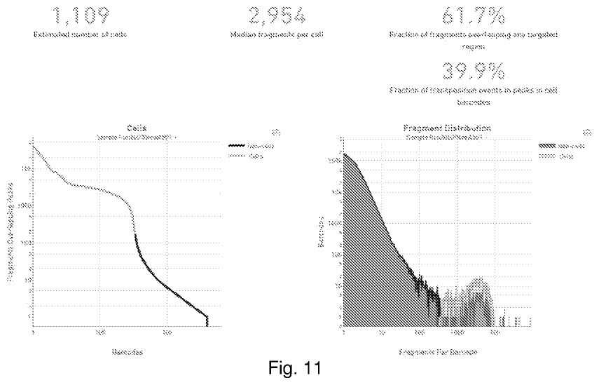

After droplet amplification, the larger droplets settle to the bottom leaving smaller droplet and oil on top. The top 504 was removed and discarded without disturbing bottom layer of settled droplets. 504 of breaking solution (100 mM NaCl, 10 mM Tris-HCl, pH 7.5, 0.2% SDS, 15% Isopropanol) was added to the emulsion and mixed 10 times. The emulsion was centrifuged for 8 minutes on a 10k mini-fuge. An additional 10-154 of the top oil layer was removed and discarded, being sure not to remove any of the bottom aqueous layer. Slowly, 604 of the bottom aqueous solution was removed from the bottom and placed in a new tube, while being careful not to aspirate any residual oil on the top layer. A 1.2× bead cleanup was performed by adding 724 of AMPure XP beads to the aqueous solution. The mixture was incubated for 5 minutes at room temperature and then placed on a magnet for 2-3 minutes (or until clear). The clear supernatant was removed and two washes using 2004 freshly prepared 80% Ethanol was performed. Washed beads were resuspended in 334 of low TE buffer. 304 was removed and placed into a new PCR tube. 154 of cleaned up products were used for a final PCR amplification in a 404 mix of 1× Phusion Hot Start II High Fidelity PCR master mix with P7 primer and one of multiplex primers from TELL-Seq Library Multiplex Primer (1-8) kit (Universal Sequencing Technology, Carlsbad, Calif.) to generate an Illumine sequencing library. The following PCR program was performed: 95° C. for 30 seconds, 5 cycles of (95° C. for 20 seconds, 63° C. for 30 seconds, and 72° C. for 30 seconds), 72° C. for 2 minutes, and hold at 4° C. A 1.2×AMPure XP bead cleanup was performed by adding 484 of AMPure XP beads to the PCR product. The mixture was incubated for 5 minutes at room temperature and then placed on a magnet for 2-3 minutes (or until clear). The clear supernatant was removed and two washes using 2004 freshly prepared 80% Ethanol was performed. Washed beads were resuspended in 254 of low TE buffer. 234 was removed and transferred into a new PCR tube. The final library was quantified using a high sensitivity D1000 screen tape on a TapeStation (FIG. 10). The library was sequenced on a NextSeq 500. Total 25,123,635 sequencing read pairs were produced. 98.5% reads pairs contained a valid barcode. Further analysis using Cell Ranger v1.2.0 identified 1,109 cells (FIG. 11) with 2,954 median fragments per cell. Knee-plot demonstrated a clear single-cell behavior.

### REFERENCES

Adey A. et al. 2010. Genome Biol. 11, R119.
Amini S. et al. 2014. Nature Genetics, 46(12):1343-1349.
Au, T. et al. 2004. EMBO J., 23: 3408-3420.
Buenrostro J. D. et al. 2013. Nature Methods, 10(12): 1213-1218.
Buenrostro, J. D. et al. 2015. Nature, 523: 486-490.
Burton B. M. and Baker T. A. 2003. Chemistry & Biology 10: 463-472.
Caruccio N. 2011. Methods Mol. Biol. 733: 241-255.
Kavanagh I, Kiiskinen L. L. and Haakana H. 2013. Unite State Patent Application Publication US2013/0023423.
Kurihara K. et al. 2011. Nat. Chem. 3: 775-781.
Laouini A. et al. 2012. Colloid Sci. Biotechnol. 1: 147-168.
Mizuuchi M., Baker T. A. and Mizuuchi K. 1992. Cell 70,303-311.
Picelli S. et al. 2014. Genome Research 24, 2033-2040.
Savilahti H., P. A. Rice, and K. MiZuuchi. 1995. EMBO J. 14:4893-4903.
Stoeckius M., et al. 2017. Nature Methods 14: 865-868.
Surette M., Buch S. J. and Chaconas G. 1987. Cell 70: 303-311.
Reznikoff W. S. 2008. Annual Review of Genetics 42(1): 269-286.

1-54. (canceled)

55. A method for barcoding a sample comprising:
a. providing a plurality of samples; each sample comprising a plurality of nucleic acid molecules
b. providing a plurality of unique barcode templates, each having a different barcode sequence;
c. compartmentalizing said plurality of samples and said plurality of barcode templates to generate a plurality of compartments, at least a portion of the plurality of compartments each comprise a single sample and at least one unique barcode template;
d. amplifying said at least one barcode template in each compartment to generate amplified barcode sequences, and attaching the amplified barcode sequences to the plurality of nucleic acid molecules comprised within each sample, thereby producing a plurality of barcode-tagged nucleic acid molecules;
e. pooling from each compartment the plurality of barcode-tagged nucleic acid molecules, thereby producing a pool of barcode-tagged nucleic acid molecules; and
f. sequencing said pool of barcode-tagged nucleic acid molecules to characterize the plurality of samples on a per sample basis.

56. The method of claim 55, wherein each barcode template comprises a central barcode sequence flanked by two handle sequences, wherein each handle sequence is configured as a priming site, a hybridization site or a binding site.

57. The method of claim 55, wherein each unique barcode template is provided in a single copy.

58. The method of claim 55, wherein each sample comprises a nucleic acid target.

59. The method of claim 58, wherein said nucleic acid target form strand transfer complexes with a plurality of transpososomes before the compartmentalizing step, wherein each transpososome comprises at least one transposon and at least one transposase; wherein said transposase is selected from the group consisting of Tn, Mu, Ty, and Tc transposases in a wildtype or a mutant or a tagged version thereof, and a combination thereof.

60. The method of claim 58, wherein said nucleic acid target is double-stranded DNA, DNA/RNA hybrid, or a combination thereof.

61. The method of claim 55, wherein each sample comprises a cell or a nucleus.

62. The method of claim 61, wherein said cell or nucleus is fixed or permeabilized before the compartmentalizing step.

63. The method of claim 61, further comprising synthesizing a cDNA in said cell or nucleus by using reverse transcriptase before the compartmentalizing step or in each compartment after the compartmentalizing step.

64. The method of claim 63, wherein said cDNA is based on a whole transcriptome, or from at least one specific target nucleic acid.

65. The method of claim 63, wherein said cDNA forms strand transfer complexes with a plurality of transpososomes, wherein each transpososome comprises at least one transposon and at least one transposase, wherein said transposase is selected from the group consisting of Tn, Mu, Ty, and Tc transposases in a wildtype or a mutant or a tagged version thereof, and a combination thereof.

66. The method of claim 63, wherein a unique molecule identifier (UMI) sequence is introduced to said cDNA.

67. The method of claim 61, wherein said cell or nucleus forms strand transfer complexes on an accessible chromatin with a plurality of transpososomes before compartmentation, wherein each transpososome comprises at least one transposon and at least one transposase, wherein said transposase is selected from the group consisting of Tn, Mu, Ty, and Tc transposases in a wildtype or a mutant or a tagged version thereof, and a combination thereof.

68. The method of claim 61, wherein said cell or nucleus form strand transfer complexes on the whole genomic DNA with a plurality of transpososomes before compartmentation, wherein each transpososome comprises at least one transposon and at least one transposase; wherein said transposase is selected from the group consisting of Tn, Mu, Ty, and Tc transposases in a wildtype or a mutant or a tagged version thereof, and a combination thereof.

69. The method of claim 61, wherein said cell or nucleus is pre-selected with one or more recognizable markers.

70. The method of claim 69, wherein said markers are identified by sequencing.

71. The method of claim 61, wherein said cell is a human cell.

72. The method of claim 61, wherein said cell is a prokaryotic cell.

73. The method of claim 55, wherein said compartmentalizing step further comprises using a water-in-oil emulsion or a liposome, wherein each compartment has a diameter from about 10 μm to about 200 μm, and preferably from about 20 μm to about 100 μm.

74. The method of claim 55, wherein said compartmentalizing step comprises physical compartmentation with a microwell, a microarray or a microtiter plate.

75. The method of claim 55, wherein the amplifying step comprises PCR, RPA, MALBAC, isothermal DNA amplification steps and template switching PCR, and a combination thereof.
